(19) United States
(12) Patent Application Publication
(10) Pub. No.: US 2015/0299779 A1
(43) Pub. Date: Oct. 22, 2015

(54) METHOD TO IDENTIFY M. TUBERCULOSIS

(71) Applicant: Vallabhbhai Patel Chest Institute, Delhi (IN)

(72) Inventors: Mandira Varma Basil, Delhi (IN); Kushal Garima, Delhi (IN); Shailendra Kumar Dhar Dwivedi, Delhi (IN); Rakesh Pathak, Delhi (IN); Mridula Bose, Delhi (IN); Rajendra Prasad, Delhi (IN); Anshika Narang, Delhi (IN); Anuj Bhatnagar, Delhi (IN)

(21) Appl. No.: 14/671,686

(22) Filed: Mar. 27, 2015

(30) Foreign Application Priority Data
Mar. 29, 2014 (IN) 911/DEL/2014

(51) Int. Cl. C12Q 1/68

(52) U.S. Cl. CPC C12Q 1/689

(57) ABSTRACT

The present invention relates to a kit and a method to identify M. tuberculosis, which may include an approach relating to isolating chromosomal DNA from a biological sample, such as a culture, conducting a real time PCR to obtain an amplicon, digesting said amplicon separately with restriction enzymes NruI and BamHI to obtain a digestion product separating said digestion product into its constituents in a gel; and analyzing the restriction enzyme digestion pattern.

### CROSS-REFERENCE REFERENCE TO RELATED APPLICATIONS

This application claims the benefit of priority to Indian Patent Application Number 911/DEL/2014, filed on Mar. 29, 2014, the contents of which is hereby incorporated by reference herein

### SEQUENCE LISTING

The present application is being filed along with a sequence listing in electronic format. The sequence listing is provided as a file entitled SeqList_ANAND013_{—}001AUS.txt, created Mar. 27, 2015 which is 1 KB in size. The information in the electronic format of the sequence listing is incorporated herein by reference in its entirety.

### FIELD OF THE INVENTION

The present invention relates to a method to identify M. tuberculosis using the restriction enzymes NruI and BamHI.

### BACKGROUND OF THE INVENTION

Tuberculosis is a major cause of death due to a single infectious agent. Early diagnosis and treatment of tuberculosis not only improve patient outcome but also helps to control this disease by reducing transmission. Unfortunately, conventional methods of bacteriological diagnosis of tuberculosis are time-consuming and labor-intensive. Moreover, although Mycobacterium tuberculosis is the most important Mycobacterium species from a public health perspective. Nontuberculous mycobacteria (NTM) are increasingly being reported in various parts of the world. Though NTM are ubiquitous organisms, normally found in the environment, several species are pathogenic to humans. Hence, rapid identification of Mycobacterium spp. is imperative for appropriate diagnosis and treatment, especially in immuno-compromised individuals.

The limitations of conventional methods of diagnosis of tuberculosis have led to the development of several nucleic acid amplification techniques which have the advantage of being rapid, sensitive and specific. However, their expense and the need for technical expertise make them difficult to be used in regions endemic for tuberculosis.

PRA techniques have been developed for several mycobacterial genes, such as hsp65, the 16S-23S rDNA spacer and rpoB. However, most of these techniques require the use of an algorithm to identify the Mycobacterium species. Also, the formation of short restricts or small differences between bands needs the use of Nusieve or Metaphor agarose which are expensive; or polyacrylamide gel, which is difficult to handle, thus making the assay technically demanding.

Accordingly, to overcome the problems encountered in the prior art, the inventors of the present invention provide a method to identify M. tuberculosis using the restriction enzymes NruI and BamHI, as described herein below.

### SUMMARY OF THE INVENTION

An object of the subject matter of the present invention is to provide a method to identify M. tuberculosis using the restriction enzymes NruI and BamHI.

Another object of the subject matter of the present invention is to provide a method to identify M. tuberculosis which is simple, cost effective, rapid, highly sensitive and specific.

Another object of the subject matter of the present invention is to provide a method to differentiate between M. tuberculosis and Non Tuberculous mycobacteria.

Another object of the subject matter of the present invention is to provide a kit to identify M. tuberculosis.

To achieve the above purposes, the present invention identifies M. tuberculosis in a subject by the following method:
isolating chromosomal DNA from a biological sample
conducting a real time PCR to obtain an amplicon
digestion of said amplicon separately with restriction enzymes NruI and BamHI to obtain a digestion product
separating said digestion product into its constituents; and
analyzing the restriction enzyme digestion pattern

The present invention also relates to a kit to identify M. tuberculosis comprising
restriction enzymes NruI and BamHI; and
a manual for providing guidelines for identification of M. tuberculosis

The subject matter will now be described in detail with reference to accompanying examples.

### BRIEF DESCRIPTION OF THE DRAWINGS

The aforementioned aspects and other features of the present invention will be explained in the following description, taken in conjunction with the accompanying drawings, wherein:

FIG. 1A depicts the digestion of PCR amplicons of reference strains of Mycobacterium Spp. with NruI. Digestion of PCR amplicons of reference strains of Mycobacterium spp. with NruI. Lane 1, 50-bp DNA marker. Lanes 2 to 17, restriction digests of H37Rv, M. bovis, M. bovis BCG, M. microti, M. smegmatis, M. simiae, M. xenopi, M. vaccae, M. malmoense, M. kansasii, M. fortuitum, M. avium, M. intracellulare, M. gordonae, M. phlei, and M. terrae, respectively. Lane 18, 50-bp marker.

FIG. 1B depicts the digestion of PCR amplicons of reference strains of Mycobacterium Spp. with BamHI. Digestion of PCR amplicons of reference strains of Mycobacterium spp. with BamHI. Lane 1, 50-bp DNA marker. Lanes 2 to 17, restriction digests of H37Rv, M. bovis, M. bovis BCG, M. microti, M. smegmatis, M. simiae, M. xenopi, M. vaccae, M. malmoense, M. kansasii, M. fortuitum, M. avium, M. intracellulare, M. gordonae, M. phlei, and M. terrae, respectively. Lane 18, 50-bp marker.

### DETAILED DESCRIPTION OF THE PREFERRED EMBODIMENT

The present invention is explained in detail below.

The inventors have developed a novel PCR Restriction Analysis (PRA) of the hsp65 gene for rapid screening of samples for M. tuberculosis complex (MTBC). Restriction enzymes NruI and BamHI were selected to obtain a limited number of restriction patterns to make differentiation between MTBC and Non Tuberculous Mycobacteria (NTM) simple. The NruI/BamHI PRA is simple as it does not require any elaborate analyses. It is cost effective, rapid, highly sensitive and specific and did not require technical expertise. The assay could, therefore, be used as a simple screening test to differentiate MTBC from NTM in peripheral laboratories.

PRA techniques have been developed for several mycobacterial genes, such as hsp65, the 16S-23S rDNA spacer and rpoB. However, most of these techniques require the use of an algorithm to identify the Mycobacterium species. Also, the formation of short restricts or small differences between bands needs the use of Nusieve or Metaphor agarose which are expensive ; or polyacrylamide gel, which is difficult to handle, thus making the assay technically demanding. The inventors of the present invention have developed a simple PRA technique to differentiate between MTBC and NTM, which does not require technical expertise and could be used as a screening assay in peripheral laboratories in tuberculosis endemic areas. It was cost effective, rapid, highly sensitive and specific. The assay could, therefore, be used as a simple screening test to differentiate MTBC from NTM in peripheral laboratories.

The method is used even in small laboratories to identify M. tuberculosis, and also to differentiate it from Non tuberculous mycobacteria. Non tuberculous mycobacteria (NTM) are ubiquitous and can be present as contaminants. They can also cause disease, especially in immuno-compromised patients. Routine smear examination cannot differentiate between MTBC and NTM. The present PRA method was evolved to make differentiation between MTBC and NTM easy not only in cultures, but also in clinical samples like sputum.

In order that this invention may be better understood the following examples are described for illustrative purposes only. The examples illustrate the present invention and are not intended to limit it in spirit or scope.

### EXAMPLES

### Materials and Methods

Clinical Isolates: A total of 310 clinical isolates were obtained from clinically suspected patients of pulmonary tuberculosis. The patients were adults ≧18 years old and were not co-infected with HIV. The clinical isolates were subjected to biochemical identification using niacin, nitrate reduction and semi-quantitative catalase tests by the standard procedure.

Reference Strains: In addition to the clinical isolates of Mycobacterium sp., 24 reference i.e. strains Mycobacterium tuberculosis (H37Rv), Mycobacterium Bovis (ATCC 19210T), Mycobacterium microti (ATCC 25584), Mycobacterium avium (MTCC, IMTECH, Chandigarh, India), Mycobacterium intracellulare (ATCC 13950), Mycobacterium gordonae (ATCC 14470), Mycobacterium fortuitum (ATCC 6841), Mycobacterium kansasii (ATCC 21982), Mycobacterium phlei (ATCC 11758), Mycobacterium smegmatis (ATCC 19420), Mycobacterium terrae (ATCC 15755), Mycobacterium vaccae (ATCC 15483), Mycobacterium malmoense (ATCC 29571), Mycobacterium xenopi (ATCC 19250), Mycobacterium simiae (ATCC 25275T), Nocardia brasiliensis (ATCC 19296), Nocardia asteroides (ATCC 19247), Escherichia coli (ATCC 35218), Klebsiella pneumoniae (ATCC 13883), Pseudomonas aeruginosa (ATCC 33348), Enterococcus faecalis (ATCC 19433), Staphylococcus aureus (ATCC 25923), Haemophilus influenzae (ATCC 35891) and Streptococcus pneumoniae (ATCCBAA-255) were also included in the study.

DNA Extraction from cultures and spiked sputum sample: Chromosomal DNA was extracted from the clinical mycobacterial isolates, the reference strain H37Rv and the reference non-tuberculous mycobacterial strains by the CTAB method as described previously. DNA was extracted from cultures of bacteria other than Mycobacterium sp. by boiling. Briefly, a loopful of mycobacterial growth was transferred to a micro-centrifuge tube containing 100 μl of 1% TritonX-100 and 50 μl of sterile double distilled water. The suspension was vortexed and boiled at 100° C. for 30 min. The suspension was centrifuged at 8000 rpm for 10 min and the clear supernatant containing mycobacterial DNA was taken for PCR.

A smear negative sputum sample was spiked with serial dilutions of H37Rv to test the lower limit of detection with the assay. DNA was extracted from the spiked sputum samples as described earlier. Briefly, equal amounts of sputum sample and 4% NaOH were mixed and incubated for 10 min at 37° C. The mixture was centrifuged at 14000 rpm for 5 min and the pellet thus obtained washed three times with PBS (pH-7.4). The pellet was finally suspended in 1% Triton X-100 and the suspension boiled at 100° C. for 30 min. The lysate was centrifuged at 8000 rpm for 10 min and the supernatant collected for PCR.

### Development of the Assay

Sequences of thirty four mycobacterial species were obtained from GenBank; and target (Sequence ID No. : 1) HSP N3: 5′-AAGAAGTGGGGTGCCCCC-3′ and (Sequence ID No. 2) antisense HSP N4: 5′-CTTGGTCTCGACCTCCTTG-3′ primers specific for hsp65 of the mycobacterial species were designed. Restriction enzymes compatible with the 300 by region of hsp65 gene of MTBC were screened by the BioEdit software so that the restricted bands were large enough to be visualized on agarose gel. Search for restriction enzymes was made so as to obtain only two bands on digestion with the enzymes to make differentiation of MTBC with NTM simple. Restriction enzymes NruI and BamHI were selected for further experiments since both the enzymes restricted the 300 by region of hsp65 gene of MTBC, whereas the NTM were restricted by either NruI or BamHI but not both.

PCR amplification

Amplification of 310 cultures of Mycobacterium sp., 24 reference strains and sputum sample spiked with varying concentrations of H37Rv was performed with the primer set HSP N3 and HSP N4, amplifying a 300 by region of the hsp65 gene. 5 μl of the extracted DNA or 7 μl of the supernatant collected from processing the spiked sputum sample were used as template for PCR. The 30 μl PCR reaction mix consisted of 10 pM of each primer, 200 μM of each deoxynucleoside triphosphate, 1.5 mM MgCl2 and 0.8 U Taq polymerase (Biotools, B&M Labs, Madrid, Spain). The thermal profile consisted of an initial denaturation for 10 min at 94° C., followed by 40 cycles of denaturation at 94° C. for 30s, annealing at 60° C. for 30s and primer extension at 72° C. for 1 min. Final extension was performed at 72° C. for 10 min. The PCR product was analyzed by electrophoresis on a 1.5% agarose gel along with a 50 by DNA ladder used as a marker.

Positive and Negative Controls Used in the PCR Assay

Each PCR run contained a positive control (H37Rv DNA) and a negative control (double distilled water). For PCR directly from spiked sputum samples, the samples were also tested for the presence of the human β-actin gene as described earlier (6). This served as an internal control in each PCR reaction.

Restriction Analysis with NruI and BamHI

Restriction digestion of the 300 by product was carried out with 1 U each of NruI and BamHI in separate tubes. Restriction digestion was carried out with 10 μl of the amplicon at 37° C. for 2 hours. The products were electrophoresed on 2.5% agarose gel with a 50 by DNA marker.

PRA using Sau96I and CfoI

To test the efficacy of the assay and confirm the test, 150 clinical mycobacterial isolates were also tested by PRA using the enzymes Sau96I and CfoI as previously described.

Sequencing

PCR products obtained by amplification of the hsp65 gene with primers HSP N3 and HSP N4 of a subset of clinical isolates (n=53) were subjected to sequencing by an ABI Automated Sequencer (Ocimum Biosolutions, Bangalore, India). Sequences were identified by similarity using Blastn available at NCBI. Species identification was confirmed if 97% match was achieved with any sequence deposited in the database, according to the criteria proposed by McNabb et al.

Sensitivity of the Assay when Used Directly on Clinical Samples

With the intent to identify M. tuberculosis directly in clinical specimens, the lower limit of detection of the assay using primers HSP N3 and HSP N4 was estimated. The sensitivity of the assay was tested on a smear negative sputum sample obtained from a patient not suffering from tuberculosis. The sample was spiked with serial dilutions of H37Rv (21) to obtain a final concentration of 10⁰, 10¹, 10², 10³, 10⁴, 10⁵ and 10⁶ organisms/μl. DNA was extracted from the spiked sputum samples as described above. The suspensions were subjected to PCR with primer sets HSP N3 and HSP N4. PRA was performed on the amplicons obtained with restriction enzymes NruI and BamHI as described previously.

Statistical Analysis

Kappa value was determined to find out the agreement between NruI/BamHI and Sau96I/CfoI assays by using GraphPad software (GraphPad, La Jolla, Calif.).

### Results

### Identification of Mycobacteria by Biochemical Reactions.

All the clinical isolates were subjected to biochemical identification by Niacin, Nitrate reduction and semi-quantitative catalase tests. Of the 310 isolates, 236 were identified as M. tuberculosis, whereas, 8 isolates were identified as NTM. However, 66 isolates could not be identified definitively on the basis of biochemical reactions.

Identification of Mycobacteria by hsp65 PRA Using the Restriction Enzymes NruI and BamHI.

The DNA obtained from 310 cultures of Mycobacterium sp. and 15 mycobacterial reference strains were used to amplify a 300 by region of the hsp65 gene using the primers HSP N3 and HSP N4. All mycobacterial isolates were amplified by the primers. PCR with primers HSP N3 and HSP N4 was also carried out with all the non-mycobacterial reference strains (viz. Nocardia brasiliensis, Nocardia asteroides, Escherichia coli, Klebsiella pneumoniae, Pseudomonas aeruginosa, Enterococcus faecalis, Staphylococcus aureus, Haemophilus influenzae and Streptococcus pneumoniae) obtained from ATCC and MTCC (IMTECH, Chandigarh). However, none of the non-mycobacterial species were amplified.

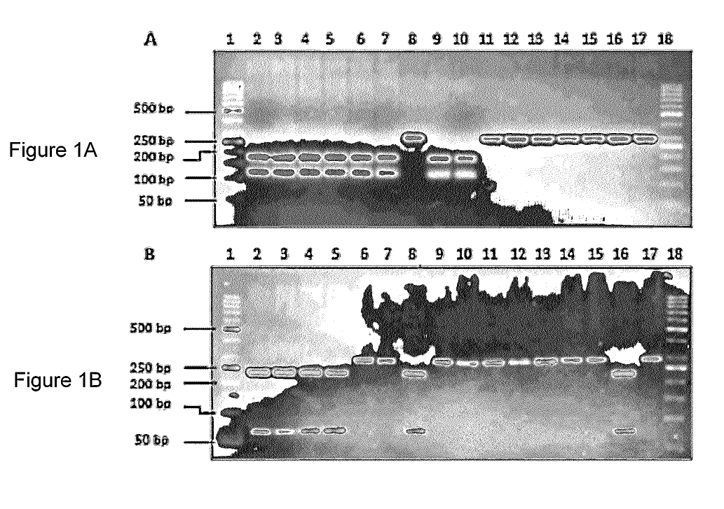

Restriction enzymes compatible with the 300 by region of hsp65 were screened. The enzyme NruI was found to restrict the amplified sequences of M. tuberculosis complex at TCG/CGA giving two bands (121 by and 179 bp) (FIG. 1A), while BamHI restricted the same sequences at G/GATCC giving two bands (66 by and 234 bp) (FIG. 1B).

We labeled the restricted pattern with Nrul as “A” and the unrestricted pattern as “a”. The restricted pattern of BamHI was labeled as “B” and the unrestricted pattern was labeled as “b” (Table 1). M. tuberculosis, M. Bovis and M. microti were the only mycobacterial species amongst all the species tested which were restricted by both NruI and BamHI (restriction pattern “AB”) (Table 2). Of all the culture isolates tested, 295 were identified as M. tuberculosis complex by NruI/BamHI PRA, including 59 of the 66 isolates that had not been identified definitively on the basis of biochemical reactions. The remaining 7/66 isolates that could not be identified definitively by conventional methods were determined to be NTM by NruI/BamHI PRA.

TABLE 1

Restriction pattern of
PCR amplicon of hsp65 gene

| Restriction Enzyme | Restricted | Not restricted |
|---|---|---|
| NruI | A | B |
| BamHI | a | b |

TABLE 2
RESTRICTION PATTERN OF MYCOBACTERIUM
SP. USING NRUI AND BAMHI.

Restriction pattern

| Mycobacterium sp. | NruI | BamHI |
|---|---|---|
| H37Rv | A | a |
| M. bovis | A | a |
| M. microti | A | a |
| M. smegmatis | A | b |
| M. simiae | A | b |
| M. xenopi | B | a |
| M. vaccae | A | b |
| M. melomoense | A | b |
| M. kansasii | B | b |
| M. fortuitum | B | b |
| M. avium | B | b |
| M. intracellularae | B | b |
| M. gordonae | B | b |
| M. phlei | B | a |
| M. terrae | B | b |

### Testing the Efficacy of NruI/BamHI PRA

To test the ability of the PRA using restriction enzymes NruI and BamHI in correctly differentiating between MTBC and NTM, 150 clinical isolates were subjected to PRA using the enzymes Sau96I and CfoI. These included all the isolates identified to be NTM. Of the 150 mycobacterial isolates, 135 were identified as MTBC by both the methods. Of the 15 isolates identified as NTM by using the enzymes NruI and BamHI, all were confirmed to be NTM by using Sau96I and CfoI also.

### Sequencing

Of the 53 isolates sequenced, thirty eight had been restricted by both NruI and BamHI and were identified as M. tuberculosis on sequencing, thus confirming our results. Of the remaining 15 clinical isolates sequenced, all had been determined to be NTM by PRA using NruI/BamHI as well as Sau96I/CfoI. The isolates were identified to be M. avium-intracellulare (n=9), M. fortuitum (n=3) or M. abscessus (n=3) by sequencing. The NruI/BamHI assay was thus, 100% concordant with sequencing (Kappa value 1).

### Sensitivity of the Assay

To detect the sensitivity of PRA using primers HSP N3 and HSP N4 and enzymes NruI and BamHI directly on sputum samples, DNA was extracted from serial dilutions of H37Rv used to spike a smear negative sputum sample to obtain a final concentration of 10°, 10¹, 10², 10³, 10⁴, 10⁵, and 10⁶ organisms/μl and used as template for hsp65 PCR. PRA was performed, on the amplicons obtained, with restriction enzymes NruI and BamHI. The lower limit of identification with BamHI was 10 organisms/μl, however, a reliable PCR restriction analysis with both NruI and BamHI was only possible down to a concentration of 10² organisms/μl.

The various embodiments described above can be combined to provide further embodiments. Aspects of the embodiments can be modified, if necessary to employ concepts of the various patents, applications and publications to provide yet further embodiments.

Although the disclosure of system and method has been described in connection with the embodiment of the present disclosure illustrated in the accompanying drawings and examples, it is not limited thereto. It will be apparent to those skilled in the art that various substitutions, modifications and changes may be made thereto without departing from the scope and spirit of the disclosure.

What is claimed is:

1. A method to identify M. tuberculosis in a subject comprising:
isolating chromosomal DNA from a biological sample (Culture);
conducting a real time PCR to obtain an amplicon;
digesting said amplicon separately with restriction enzymes NruI and BamHI to obtain a digestion product;
separating said digestion product into its constituents; and
analyzing the restriction enzyme digestion pattern

2. The method as claimed in claim 1, wherein said sample is a sputum sample.

3. The method as claimed in claim 1, wherein the primers for conducting said PCR is:
Sequence ID 1: 5′-AAGAAGTGGGGTGCCCCC-3′
or
Sequence ID 2: 5′-CTTGGTCTCGACCTCCTTG-3′

4. The method as claimed in claim 1, wherein the digestion product is separated into its constituents by agarose gel electrophoresis.

5. The method as claimed in claim 1, wherein said method further comprises identifying the number of digests.

6. A kit to identify M. tuberculosis comprising:
restriction enzymes NruI and BamHI; and
a manual for providing guidelines for identification of M. tuberculosis.


SEQUENCE LISTING (2 sequences)

SEQ ID NO: 1 - DNA, 18 bp, Artificial Sequence
  Primer
aagaagtggggtgccccc

SEQ ID NO: 2 - DNA, 19 bp, Artificial Sequence
  Primer
cttggtctcgacctccttg